(19) United States
(12) Patent Application Publication
(10) Pub. No.: US 2012/0070401 A1
(43) Pub. Date: Mar. 22, 2012

(54) Composition and Method for Promoting Wound Healing

(76) Inventors: Jinzhong Zhang, Pittsford, NY (US); Keith W. Ward, Ontario, NY (US); Francisco J. Lopez, Victor, NY (US); Matthew Jonasse, Webster, NY (US)

(21) Appl. No.: 13/217,336

(22) Filed: Aug. 25, 2011

Related U.S. Application Data
(60) Provisional application No. 61/384,754, filed on Sep. 21, 2010.

(51) Int. Cl. A61K 31/79; A61K 31/734; A61K 31/728; A61K 31/722; A61K 31/78; A61P 27/02; A61P 31/10; A61P 33/02; A61P 29/00; A61P 31/04; A61P 37/06; A61K 31/717; A61P 31/12

(52) U.S. Cl. 424/78.04; 424/78.06; 514/57; 514/54; 514/55

(57) ABSTRACT

A composition for promoting wound healing comprises a bioadhesive polymer and a pharmaceutically acceptable liquid medium. Such a composition is applied to a wound to promote healing of the wound. The bioadhesive polymer can be selected from the group consisting of natural or synthetic hydrophilic polymers and hydrogels.

### CROSS REFERENCE

This application claims the benefit of Provisional Patent Application No. 61/384,754 filed Sep. 21, 2010, which is incorporated by reference herein.

### BACKGROUND

The present invention relates to a composition and method for promoting wound healing. In particular, the present invention relates to a composition comprising a bioadhesive polymer, and a method using such composition, for healing wounds.

The process of wound healing involves a highly orchestrated series of biological responses to tissue injury, including proliferation, migration, and extracellular matrix deposition1-5. In the cornea, wound recovery begins with epithelial cell migration, which occurs independent of epithelial cell proliferation until the wounded area is closed⁶. Thereafter, the thickness of the epithelial layer is recovered with a combination of new cell proliferation and movement of cells into the new epithelial layer from the basal layer. While in vitro models can recapitulate most of these individual processes, and can be informative in the study of wound healing from a mechanistic point of view, such a complex process can best be studied in vivo, and there are numerous available literature examples of relevant corneal wound healing models⁷⁻⁹. Antimicrobial agents have been used to help the wound healing process have been used. However, there is still a need to provide simple, non-toxic, easy-to-use materials to assist wound healing.

### SUMMARY

In general, the present invention provides a composition comprising a bioadhesive polymer for use to promote wound healing, and a method for healing wound by applying such a composition to a wound. A bioadhesive polymer is a natural or synthetic polymer that has a property of adhering to a biological surface upon being applied thereto.

In one aspect, a composition of the present invention comprises a bioadhesive polymer and a pharmaceutically acceptable vehicle.

In another aspect, a composition of the present invention comprises a bioadhesive polymer and a pharmaceutically acceptable vehicle, wherein the bioadhesive polymer comprises a mucoadhesive adhesive, and the pharmaceutically acceptable vehicle comprises a liquid medium. The term “mucoadhesive polymer” means a natural or synthetic polymer that has a property of adhering to a mucosal surface upon being applied thereto

In still another aspect, a composition of the present invention comprises a bioadhesive polymer and a pharmaceutically acceptable vehicle, wherein the bioadhesive polymer comprises a mucoadhesive polymer, and the pharmaceutically acceptable vehicle comprises a liquid medium.

In yet another aspect, a composition of the present invention comprises a bioadhesive polymer and a pharmaceutically acceptable vehicle, wherein the bioadhesive polymer comprises a mucoadhesive polymer, and the pharmaceutically acceptable vehicle comprises an aqueous medium. The term “aqueous medium” means a medium comprising water, and optionally other materials, which may be soluble in water or in another material included in such medium.

In a further aspect, the present invention provides a method for promoting wound healing, the method comprising applying to a wound a composition that comprises a bioadhesive polymer and a pharmaceutically acceptable vehicle, wherein the pharmaceutically acceptable vehicle comprises an aqueous medium.

Other features and advantages of the present invention will become apparent from the following detailed description and claims and the appended drawings.

### DESCRIPTION OF THE DRAWINGS

FIG. 1. Representative images showing the process of corneal reepithelialization in the Besivance® group. Slit-lamp photographs were taken after instillation of topical fluorescein drops. Image acquired immediately following surgery (Left), 36 hours post-surgery (Middle), and 72 hours post-surgery (Right), respectively. The dotted ring (Left or Middle) surrounding the fluorescein-labeled area represents the wound area of the cornea.

FIG. 2. Representative histochemical sections of the rabbit cornea stained with hematoxylin and eosin. Image on the left shows a multilayered epithelium acquired from an unwounded area of the cornea from an animal in the saline group. The image in the middle shows an unclosed corneal defect at 72 hours from an animal in the Vigamox® group. The arrow points to the epithelial front as the wound is healing. The image on the right shows the closed wound from an animal in the Besivance® group at 72 hours post-surgery, exhibiting a reduced number of corneal cell layers as compared with the intact epithelium shown in the left panel. Scale bar, 50 μm.

FIG. 3. Besivance® and other fluoroquinolones do not affect corneal wound area in a rabbit wound healing model. Data are means±SEM, n=10 per group. Data were analyzed by a two-way ANOVA with repeated measures followed by the Tukey-Kramer test on data elevated to the power 0.6 prior to statistical analysis.

FIG. 4. Integrated effects of Besivance® and other fluoroquinolones on the corneal wound area in a rabbit wound healing model. Integrated responses were analyzed by calculating the areas under the curve using the trapezoidal rule for each treatment over the time course. Data are means±SEM, n=10 per group. Data were analyzed by a one-way ANOVA followed by the Tukey-Kramer test on raw data.

FIG. 5. DuraSite® reduces wound area in a rabbit wound healing model. Data are means±SEM, n=10 per group. Data were analyzed by a two-way ANOVA with repeated measures followed by the Tukey-Kramer test on data elevated to the power 0.6 prior to statistical analysis. *represents time points at which the DuraSite® group was significantly different from the saline group. P<0.05.

FIG. 6. Integrated effects of 100 ppm BAK, DuraSite®, Durasite®+100 ppm BAK, and AzaSite® on wound area in a rabbit wound healing model. Integrated responses were analyzed by calculating the areas under the curve using the trapezoidal rule for each treatment over the time course. Data are means±SEM, n=10 per group. Data were analyzed by a one-way ANOVA followed by the Tukey-Kramer test on raw data. *vs. Saline; P<0.05.

FIG. 7. BAK does not affect corneal wound area in a rabbit wound healing model. Data are means±SEM. n=10 per group. Data were analyzed by a two-way ANOVA with repeated measures followed by the Tukey-Kramer test on data elevated to the power 0.6 prior to statistical analysis.

FIG. 8. Integrated effects of BAK on wound area in a rabbit wound healing model. Integrated responses were analyzed by calculating the areas under the curve using the trapezoidal rule for each treatment over the time course. Data are means±SEM, n=10 per group. Data were analyzed by a one-way ANOVA followed by the Tukey-Kramer test on raw data.

### DETAILED DESCRIPTION

In one aspect, a composition of the present invention comprises a bioadhesive polymer and a pharmaceutically acceptable vehicle.

In another aspect, a composition of the present invention comprises a bioadhesive polymer and a pharmaceutically acceptable vehicle, wherein the bioadhesive polymer comprises a mucoadhesive adhesive, and the pharmaceutically acceptable vehicle comprises a liquid medium.

In still another aspect, a composition of the present invention comprises a bioadhesive polymer and a pharmaceutically acceptable vehicle, wherein the bioadhesive polymer comprises a mucoadhesive polymer, and the pharmaceutically acceptable vehicle comprises a liquid medium.

In yet another aspect, a composition of the present invention comprises a bioadhesive polymer and a pharmaceutically acceptable vehicle, wherein the bioadhesive polymer comprises a mucoadhesive polymer, and the pharmaceutically acceptable vehicle comprises an aqueous medium.

In a further aspect, a composition of the present invention comprises a bioadhesive polymer and a pharmaceutically acceptable aqueous medium, wherein the bioadhesive polymer is suspended or dissolved in the aqueous medium, and wherein the bioadhesive polymer is selected from the group consisting of hydrophilic polymers, hydrogels, and mixtures thereof.

In one embodiment, a composition of the present invention comprises a bioadhesive polymer and a pharmaceutically acceptable aqueous medium; wherein the bioadhesive polymer is suspended or dissolved in the aqueous medium; and the bioadhesive polymer is selected from the group consisting of polyvinylpyrrolidone, methyl cellulose, carboxymethyl cellulose and its salts, hydroxypropylmethyl cellulose, other hydrophilic cellulose derivatives, alginate and its salts, hyaluronic acid and its salts, and chitosan and derivatives thereof.

In another embodiment, a composition of the present invention comprises a bioadhesive polymer and a pharmaceutically acceptable aqueous medium; wherein the bioadhesive polymer is suspended or dissolved in the aqueous medium; and the bioadhesive polymer is selected from the group consisting of lightly crosslinked poly(acrylic acid), polyvinylpyrrolidone, methyl cellulose, carboxymethyl cellulose and its salts, hydroxypropylmethyl cellulose, other hydrophilic cellulose derivatives, alginate and its salts, hyaluronic acid and its salts, and chitosan; and wherein the amount of the bioadhesive polymer is in the range from about 0.001 to about 10 percent by weight of the total composition.

As used herein, the term “lightly crosslinked” with respect to a polymer means that the amount of a crosslinking agent in such polymer is in the range from about 0.001 to about 10 percent by weight of the polymer. Alternatively, the amount of a crosslinking agent in such polymer is in the range from about 0.01 to about 5 percent (or from about 0.01 to about 2, or from about 0.1 to about 2, or from about 0.1 to about 1, from about 0.5 to about 2, or from about 1 to about 5, or from about 2 to about 5 percent) by weight of the polymer.

In one aspect, a composition of the present invention comprises a bioadhesive polymer and a pharmaceutically acceptable aqueous medium; wherein the bioadhesive polymer is suspended or dissolved in the aqueous medium; and the bioadhesive polymer comprises a lightly crosslinked poly(acrylic acid) in an amount in the range from about 0.1 to about 7 percent by weight of the total composition.

In one aspect, a composition of the present invention comprises a bioadhesive polymer and a pharmaceutically acceptable aqueous medium; wherein the bioadhesive polymer is suspended or dissolved in the aqueous medium; and the bioadhesive polymer comprises a lightly crosslinked poly(acrylic acid) in an amount in the range from about 0.1 to about 7 percent by weight of the total composition; wherein the crosslinked poly(acrylic acid) is selected from the group consisting of polycarbophil (poly(acrylic acid) crosslinked with divinyl glycol, such as Noveon® AA-1 USP), Carpobol® polymers (such as Carpobol® 974P NF, 971P NF, 71G NF, or 934P NF crosslinked with allyl ethers of pentaerythritol or allyl ethers of sucrose), and poly(C10-30 alkyl acrylate/acrylic acid) crosspolymers (such as Permulen® TR-1).

In another aspect, any composition as disclosed herein has a viscosity in the range from about 2 to about 2,000 centipoises (or mPa·s), as measured by a Brookfield viscometer (Model RVDV III) at 25° C. and a shear rate of 1-7 sec⁻¹, with a CPE-40 spindle.

In still another aspect, a composition of the present invention comprises a bioadhesive polymer and a pharmaceutically acceptable aqueous medium; wherein the bioadhesive polymer is suspended or dissolved in the aqueous medium; and the bioadhesive polymer comprises a polycarbophil in an amount in the range from about 0.1 to about 7 percent by weight of the total composition; wherein the composition has a viscosity in the range from about 3 to about 1500 mPa·s, as measured at the condition disclosed herein.

In still another aspect, a composition of the present invention comprises: (a) a bioadhesive polymer; (2) a tonicity agent (non-ionic or ionic); (3) a pharmaceutically acceptable preservative; and (4) a pharmaceutically acceptable aqueous medium; wherein the bioadhesive polymer is suspended or dissolved in the aqueous medium; and the bioadhesive polymer comprises a polycarbophil in an amount in the range from about 0.1 to about 7 percent by weight of the total composition; wherein the composition has a viscosity in the range from about 3 to about 1500 mPa·s, as measured at the condition disclosed herein.

In yet another aspect, a composition of the present invention comprises: (a) a bioadhesive polymer; (2) a tonicity agent (non ionic or inonic); (3) a pharmaceutically acceptable preservative; (4) a preservative-enhancing material; and (5) a pharmaceutically acceptable aqueous medium; wherein the bioadhesive polymer is suspended or dissolved in the aqueous medium; and the bioadhesive polymer comprises a polycarbophil in an amount in the range from about 0.1 to about 7 percent by weight of the total composition; wherein the composition has a viscosity in the range from about 3 to about 1500 mPa·s, as measured at the condition disclosed herein; wherein the preservative-enhancing material is selected from the group consisting of D-glucose, sucrose, maltose, D-mannose, trehalose, glutamic acid, mixtures thereof, and combinations thereof.

In yet another aspect, a composition of the present invention comprises: (a) a bioadhesive polymer; (2) a tonicity agent (non ionic or inonic); (3) a pharmaceutically acceptable preservative; (4) a preservative-enhancing material; (5) a chelating agent; and (6) a pharmaceutically acceptable aqueous medium; wherein the bioadhesive polymer is suspended or dissolved in the aqueous medium; and the bioadhesive polymer comprises a polycarbophil in an amount in the range from about 0.1 to about 7 percent by weight of the total composition; wherein the composition has a viscosity in the range from about 3 to about 1500 mPa·s, as measured at the condition disclosed herein; wherein the preservative-enhancing material is selected from the group consisting of D-glucose, sucrose, maltose, D-mannose, trehalose, glutamic acid, mixtures thereof, and combinations thereof.

In another embodiment the present invention, a composition comprises: (a) a bioadhesive polymer; (2) a tonicity agent (non ionic or inonic); (3) a pharmaceutically acceptable preservative; (4) a preservative-enhancing material; (5) a chelating agent; and (6) a pharmaceutically acceptable aqueous medium; wherein the bioadhesive polymer is suspended or dissolved in the aqueous medium; and the bioadhesive polymer comprises chitosan or a derivative thereof, in an amount in the range from about 0.1 to about 7 percent by weight of the total composition; wherein the composition has a viscosity in the range from about 3 to about 1500 mPa·s, as measured at the condition disclosed herein; wherein the preservative-enhancing material is selected from the group consisting of D-glucose, sucrose, maltose, D-mannose, trehalose, glutamic acid, mixtures thereof, and combinations thereof.

Furthermore, any one composition herein disclosed can further comprises an active pharmaceutical ingredient (or therapeutic agent) such as anti-inflammatory agents, antibiotics, immunosuppressive agents, antiviral agents, antifungal agents, antiprotozoal agents, combinations thereof, or mixtures thereof. Non-limiting examples of anti-inflammatory agents include glucocorticosteroids (e.g., for short-term treatment) and non-steroidal anti-inflammatory drugs (“NSAIDs”).

Non-limiting examples of the glucocorticosteroids are: 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, their physiologically acceptable salts, derivatives thereof, combinations thereof, and mixtures thereof. In one embodiment, the therapeutic agent is selected from the group consisting of difluprednate, loteprednol etabonate, prednisolone, combinations thereof, and mixtures thereof.

Non-limiting examples of the NSAIDs are: aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (e.g., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lornoxicam, piroxicam, tenoxicam), ε-acetamidocaproic acid, S-(5′-adenosyl)-L-methionine, 3-amino-4-hydroxybutyric acid, amixetrine, bendazac, benzydamine, α-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, their physiologically acceptable salts, combinations thereof, and mixtures thereof.

Non-limiting examples of antibiotics include doxorubicin; aminoglycosides (e.g., amikacin, apramycin, arbekacin, bambermycins, butirosin, dibekacin, dihydrostreptomycin, fortimicin(s), gentamicin, isepamicin, kanamycin, micronomicin, neomycin, neomycin undecylenate, netilmicin, paromomycin, ribostamycin, sisomicin, spectinomycin, streptomycin, tobramycin, trospectomycin), amphenicols (e.g., azidamfenicol, chloramphenicol, florfenicol, thiamphenicol), ansamycins (e.g., rifamide, rifampin, rifamycin SV, rifapentine, rifaximin), β-lactams (e.g., carbacephems (e.g., loracarbef)), carbapenems (e.g., biapenem, imipenem, meropenem, panipenem), cephalosporins (e.g., cefaclor, cefadroxil, cefamandole, cefatrizine, cefazedone, cefazolin, cefcapene pivoxil, cefclidin, cefdinir, cefditoren, cefepime, cefetamet, cefixime, cefinenoxime, cefodizime, cefonicid, cefoperazone, ceforamide, cefotaxime, cefotiam, cefozopran, cefpimizole, cefpiramide, cefpirome, cefpodoxime proxetil, cefprozil, cefroxadine, cefsulodin, ceftazidime, cefteram, ceftezole, ceftibuten, ceftizoxime, ceftriaxone, cefuroxime, cefuzonam, cephacetrile sodium, cephalexin, cephaloglycin, cephaloridine, cephalosporin, cephalothin, cephapirin sodium, cephradine, pivcefalexin), cephamycins (e.g., cefbuperazone, cefinetazole, cefininox, cefotetan, cefoxitin), monobactams (e.g., aztreonam, carumonam, tigemonam), oxacephems, flomoxef, moxalactam), penicillins (e.g., amdinocillin, amdinocillin pivoxil, amoxicillin, ampicillin, apalcillin, aspoxicillin, azidocillin, azlocillin, bacampicillin, benzylpenicillinic acid, benzylpenicillin sodium, carbenicillin, carindacillin, clometocillin, cloxacillin, cyclacillin, dicloxacillin, epicillin, fenbenicillin, floxacillin, hetacillin, lenampicillin, metampicillin, methicillin sodium, mezlocillin, nafcillin sodium, oxacillin, penamecillin, penethamate hydriodide, penicillin G benethamine, penicillin G benzathine, penicillin G benzhydrylamine, penicillin G calcium, penicillin G hydrabamine, penicillin G potassium, penicillin G procaine, penicillin N, penicillin O, penicillin V, penicillin V benzathine, penicillin V hydrabamine, penimepicycline, phenethicillin potassium, piperacillin, pivampicillin, propicillin, quinacillin, sulbenicillin, sultamicillin, talampicillin, temocillin, ticarcillin), lincosamides (e.g., clindamycin, lincomycin), macrolides (e.g., azithromycin, carbomycin, clarithromycin, dirithromycin, erythromycin, erythromycin acistrate, erythromycin estolate, erythromycin glucoheptonate, erythromycin lactobionate, erythromycin propionate, erythromycin stearate, josamycin, leucomycins, midecamycins, miokamycin, oleandomycin, primycin, rokitamycin, rosaramicin, roxithromycin, spiramycin, troleandomycin), polypeptides (e.g., amphomycin, bacitracin, capreomycin, colistin, enduracidin, enviomycin, fusafungine, gramicidin S, gramicidin(s), mikamycin, polymyxin, pristinamycin, ristocetin, teicoplanin, thiostrepton, tuberactinomycin, tyrocidine, tyrothricin, vancomycin, viomycin, virginiamycin, zinc bacitracin), tetracyclines (e.g., apicycline, chlortetracycline, clomocycline, demeclocycline, doxycycline, guamecycline, lymecycline, meclocycline, methacycline, minocycline, oxytetracycline, penimepicycline, pipacycline, rolitetracycline, sancycline, tetracycline), and others (e.g., cycloserine, mupirocin, tuberin).

Other examples of antibiotics are the synthetic antibacterials, such as 2,4-diaminopyrimidines (e.g., brodimoprim, tetroxoprim, trimethoprim), nitrofurans (e.g., furaltadone, furazolium chloride, nifuradene, nifuratel, nifurfoline, nifurpirinol, nifurprazine, nifurtoinol, nitrofurantoin), quinolones and analogs (e.g., cinoxacin, ciprofloxacin, clinafloxacin, difloxacin, enoxacin, fleroxacin, flumequine, grepafloxacin, lomefloxacin, miloxacin, nadifloxacin, nalidixic acid, norfloxacin, ofloxacin, oxolinic acid, pazufloxacin, pefloxacin, pipemidic acid, piromidic acid, rosoxacin, rufloxacin, sparfloxacin, temafloxacin, tosufloxacin, trovafloxacin), sulfonamides (e.g., acetyl sulfamethoxypyrazine, benzylsulfamide, chloramine-B, chloramine-T, dichloramine T, n²-formylsulfisomidine, n⁴-β-D-glucosylsulfanilamide, mafenide, 4′-(methylsulfamoyl)sulfanilanilide, noprylsulfamide, phthalylsulfacetamide, phthalylsulfathiazole, salazosulfadimidine, succinylsulfathiazole, sulfabenzamide, sulfacetamide, sulfachlorpyridazine, sulfachrysoidine, sulfacytine, sulfadiazine, sulfadicramide, sulfadimethoxine, sulfadoxine, sulfaethidole, sulfaguanidine, sulfaguanol, sulfalene, sulfaloxic acid, sulfamerazine, sulfameter, sulfamethazine, sulfamethizole, sulfamethomidine, sulfamethoxazole, sulfamethoxypyridazine, sulfametrole, sulfamidochrysoidine, sulfamoxole, sulfanilamide, 4-sulfanilamidosalicylic acid, n⁴-sulfanilylsulfanilamide, sulfanilylurea, n-sulfanilyl-3,4-xylamide, sulfanitran, sulfaperine, sulfaphenazole, sulfaproxyline, sulfapyrazine, sulfapyridine, sulfasomizole, sulfasymazine, sulfathiazole, sulfathiourea, sulfatolamide, sulfisomidine, sulfisoxazole) sulfones (e.g., acedapsone, acediasuifone, acetosulfone sodium, dapsone, diathymosulfone, glucosulfone sodium, solasulfone, succisulfone, sulfanilic acid, p-sulfanilylbenzylamine, sulfoxone sodium, thiazolsulfone), and others (e.g., clofoctol, hexedine, methenamine, methenamine anhydromethylene citrate, methenamine hippurate, methenamine mandelate, methenamine sulfosalicylate, nitroxoline, taurolidine, xibomol).

Non-limiting examples of immunosuppressive agents include dexamethasone, cyclosporin A, azathioprine, brequinar, gusperimus, 6-mercaptopurine, mizoribine, rapamycin, tacrolimus (FK-506), folic acid analogs (e.g., denopterin, edatrexate, methotrexate, piritrexim, pteropterin, Tomudex®, trimetrexate), purine analogs (e.g., cladribine, fludarabine, 6-mercaptopurine, thiamiprine, thiaguanine), pyrimidine analogs (e.g., ancitabine, azacitidine, 6-azauridine, camiofur, cytarabine, doxifluridine, emitefur, enocitabine, floxuridine, fluorouracil, gemcitabine, tegafur), fluocinolone, triaminolone, anecortave acetate, fluorometholone, medrysone, and prednisolone.

Non-limiting examples of antifungal agents include polyenes (e.g., amphotericin B, candicidin, dermostatin, filipin, fungichromin, hachimycin, hamycin, lucensomycin, mepartricin, natamycin, nystatin, pecilocin, perimycin), azaserine, griseofulvin, oligomycins, neomycin undecylenate, pyirolnitrin, siccanin, tubercidin, viridin, allylamines (e.g., butenafine, naftifine, terbinafine), imidazoles (e.g., bifonazole, butoconazole, chlordantoin, chlomiidazole, cloconazole, clotrimazole, econazole, enilconazole, fenticonazole, flutrimazole, isoconazole, ketoconazole, lanoconazole, miconazole, omoconazole, oxiconazole nitrate, sertaconazole, sulconazole, tioconazole), thiocarbamates (e.g., tolciclate, tolindate, tolnaftate), triazoles (e.g., fluconazole, itraconazole, saperconazole, terconazole), acrisorcin, amorolfine, biphenamine, bromosalicylchloranilide, buclosamide, calcium propionate, chlorphenesin, ciclopirox, cloxyquin, coparaffinate, diamthazole dihydrochloride, exalamide, flucytosine, halethazole, hexetidine, loflucarban, nifuratel, potassium iodide, propionic acid, pyrithione, salicylanilide, sodium propionate, sulbentine, tenonitrozole, triacetin, ujothion, undecylenic acid, and zinc propionate.

Non-limiting examples of antiviral agents include acyclovir, carbovir, famciclovir, ganciclovir, penciclovir, and zidovudine.

Non-limiting examples of antiprotozoal agents include pentamidine isethionate, quinine, chloroquine, and mefloquine.

In one aspect, the amount of a therapeutic agent is in the range from 0.001 to 10 percent (or alternatively, from 0.005 to 5, or 0.01 to 2, or 0.01 to 1, or 0.01 to 0.5, or 0.1 to 0.5, or 0.1 to 1, or 0.1 to 2, or 0.5 to 2, or 0.5 to 5 percent) by weight of the pharmaceutical composition.

A suitable pharmaceutically acceptable preservative for any one of the compositions herein disclosed is selected from the group consisting of pharmaceutically acceptable alcohols, amines and ammonium-containing compounds, hydrogen peroxide and compounds that produce hydrogen peroxide in said composition (such as carbamide peroxide, carbamide perhydrate, percarbamide, or perborate salts), oxychloro compounds such as chlorine dioxide, zinc compounds, mixtures thereof, and combinations thereof.

In another aspect, the pharmaceutically acceptable preservative is include in a composition of the present invention in an amount that does not produce irritation or discomfort to an eye of an average patient when such composition is administered thereto.

Non-limiting examples of ammonium-containing compounds include benzalkonium chloride (“BAK”), benzododecinium bromide (“BDD”), chlorhexidine, polymeric biguanide (such as polyhexamethylene biguanide or “PHMB”), polyquaternium-1 (also known as polidronium chloride, formula shown below), polyquaternium-4 (hydroxyethylcellulose dimethyl-diallyl ammonium chloride copolymer, sometimes known under the tradename of Celquat® H-100 or Celquat® L-200), and polyquaternium-42 (formula shown below).

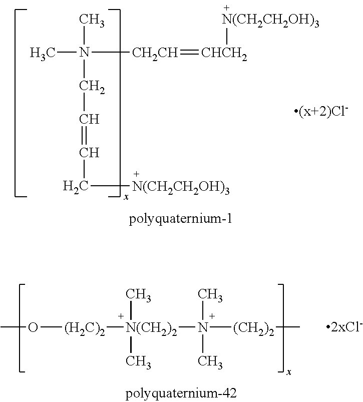

Other polyquaternium compounds, which are described in International Cosmetic Ingredient Dictionary and Handbook, can also be used in a composition of the present invention. Non-limiting examples of such other polyquaternium compounds are polyquaternium-2, -5, -6, -7, -8, -9, -45, -54, -71, and -72.

Typical alcohol-based anti-microbial agents include benzyl alcohol, phenethyl alcohol, and chlorbutanol.

In one embodiment, a composition of the present invention is free of a material selected from the group consisting of organic nitrogen-containing compounds containing a plurality of positive charges, such as organic nitrogen-containing small molecules or polymers or alcohols containing a plurality of positive charges.

A composition of the present invention can be formulated in a physiologically acceptable buffer to regulate pH and tonicity in a range compatible with ophthalmic uses and with any active ingredients present therein. Non-limiting examples of physiologically acceptable buffers include phosphate buffer; a Tris-HCl buffer (comprising tris(hydroxymethyl)aminomethane and HCl); buffers based on HEPES (N-{2-hydroxyethyl}peperazine-N′-{2-ethanesulfonic acid}) having pKₐ of 7.5 at 25° C. and pH in the range of about 6.8-8.2; BES (N,N-bis{2-hydroxyethyl}2-aminoethanesulfonic acid) having pKₐ of 7.1 at 25° C. and pH in the range of about 6.4-7.8; MOPS (3-{N-morpholino}propanesulfonic acid) having pKₐ of 7.2 at 25° C. and pH in the range of about 6.5-7.9; TES (N-tris{hydroxymethyl}-methyl-2-aminoethanesulfonic acid) having pKₐ of 7.4 at 25° C. and pH in the range of about 6.8-8.2; MOBS (4-{N-morpholino}butanesulfonic acid) having pKₐ of 7.6 at 25° C. and pH in the range of about 6.9-8.3; DIPSO (3-(N,N-bis{2-hydroxyethyl}amino)-2-hydroxypropane)) having pKₐ of 7.52 at 25° C. and pH in the range of about 7-8.2; TAPSO (2-hydroxy-3{tris(hydroxymethyl)methylamino}-1-propanesulfonic acid)) having pKₐ of 7.61 at 25° C. and pH in the range of about 7-8.2; TAPS ({(2-hydroxy-1,1-bis(hydroxymethyl)ethyl)amino}-1-propanesulfonic acid)) having pKₐ of 8.4 at 25° C. and pH in the range of about 7.7-9.1; TABS (N-tris(hydroxymethyl)methyl-4-aminobutanesulfonic acid) having pKₐ of 8.9 at 25° C. and pH in the range of about 8.2-9.6; AMPSO (N-(1,1-dimethyl-2-hydroxyethyl)-3-amino-2-hydroxypropanesulfonic acid)) having pKₐ of 9.0 at 25° C. and pH in the range of about 8.3-9.7; CHES (2-cyclohexylamino)ethanesulfonic acid) having pKₐ of 9.5 at 25° C. and pH in the range of about 8.6-10.0; CAPSO (3-(cyclohexylamino)-2-hydroxy-1-propanesulfonic acid) having pKₐ of 9.6 at 25° C. and pH in the range of about 8.9-10.3; or CAPS (3-(cyclohexylamino)-1-propane sulfonic acid) having pKₐ of 10.4 at 25° C. and pH in the range of about 9.7-11.1.

While the buffer itself is a “tonicity adjusting agent” and a “pH adjusting agent” that broadly maintains the ophthalmic solution at a particular ion concentration and pH, additional “tonicity adjusting agents” can be added to adjust the final tonicity of the solution. Such tonicity adjusting agents are well known to those of skill in the art and include, but are not limited to, mannitol, sorbitol, dextrose, sucrose, urea, propylene glycol, and glycerin. Also, various salts, including halide salts of a monovalent cation (e.g., NaCl or KCl) can be utilized.

The tonicity adjusting agent, when present, can be in a concentration ranging from about 0.01 to about 10, or from about 0.01 to about 7, or from about 0.01 to about 5, or from about 0.1 to about 2, or from about 0.1 to about 1 percent by weight. In some embodiments where a tonicity adjusting agent is present the solution can contain a single agent or a combination of different tonicity adjusting agents. Typically, the tonicity of a formulation of the present invention is in the range from about 200 to 400 mOsm/kg. Alternatively, the tonicity of a formulation of the present invention is in the range from about 220 to 400 mOsm/kg, or from about 220 to 350 mOsm/kg, or from about 220 to 300 mOsm/kg, or from about 250 to 350 mOsm/kg, or from about 250 to 300 mOsm/kg, or from about 240 to 280 mOsm/kg. For ocular wound applications, an ophthalmic formulation of the present invention may be desirably hypotonic, such as having tonicity in the range from about 200 to about 270 mOsm/kg.

Compositions of the present invention also can comprise one or more surfactants. Suitable surfactants can include cationic, anionic, non-ionic or amphoteric surfactants. Preferred surfactants are neutral or nonionic surfactants. Non-limiting examples of surfactants suitable for a formulation of the present invention include polysorbates (such as polysorbate 80 (polyoxyethylene sorbitan monooleate), polysorbate 60 (polyoxyethylene sorbitan monostearate), polysorbate 20 (polyoxyethylene sorbitan monolaurate), commonly known by their trade names of Tween® 80, Tween® 60, Tween® 20), poloxamers (synthetic block polymers of ethylene oxide and propylene oxide, such as those commonly known by their trade names of Pluronic®; e.g., Pluronic® F127 or Pluronic® F108)), or poloxamines (synthetic block polymers of ethylene oxide and propylene oxide attached to ethylene diamine, such as those commonly known by their trade names of Tetronic®; e.g., Tetronic® 1508 or Tetronic® 908, etc., other nonionic surfactants such as Brij®, Myrj®, and long chain fatty alcohols (i.e., oleyl alcohol, stearyl alcohol, myristyl alcohol, docosohexanoyl alcohol, etc.) with carbon chains having about 12 or more carbon atoms (e.g., such as from about 12 to about 24 carbon atoms). Such compounds are delineated in Martindale, 34^{th} ed., pp 1411-1416 (Martindale, “The Complete Drug Reference,” S. C. Sweetman (Ed.), Pharmaceutical Press, London, 2005) and in Remington, “The Science and Practice of Pharmacy,” 21^{st} Ed., pp 291 and the contents of chapter 22, Lippincott Williams & Wilkins, New York, 2006. The concentration of a non-ionic surfactant, when present, in a composition of the present invention can be in the range from about 0.001 to about 5 weight percent (or alternatively, from about 0.01 to about 4, or from about 0.01 to about 2, or from about 0.01 to about 1 weight percent).

In some embodiments, compositions of this invention can optionally include other viscosity adjusting agents (e.g., particularly when the ophthalmic solution is intended to act as a lubricant (i.e., artificial tear)). Suitable viscosity adjusting agents for administration to an eye are well known to those of skill in the art. One or more polysaccharides disclosed above can act as viscosity adjusting agents. Other non-ionic polysaccharides such as cellulose derivatives are commonly used to increase viscosity, and as such, can offer other advantages. Specific cellulose derivatives include, but are not limited to hydroxypropyl methyl cellulose, carboxymethyl cellulose, methyl cellulose, or hydroxyethyl cellulose. Typically, particularly when used as an artificial tear, the ophthalmic solution has a viscosity from about 1 to about 1000 centipoises (or mPa·s). As a solution, the present pharmaceutical formulation is usually dispensed in the eye in the form of an eye drop. It should be understood, however, that the present pharmaceutical formulation may also be formulated as a viscous liquid (e.g., viscosities from 50 to several thousand cps), gel, or ointment, which has even higher viscosity, for ophthalmic or non-ophthalmic uses. Furthermore, in some contact-lens related embodiments, lenses may be soaked or otherwise exposed to a pharmaceutical formulation of the present invention prior to wear.

In some embodiments, an ophthalmic formulation of the present invention can further comprise a demulcent. Polysaccharides, such as those disclosed herein above can act as demulcents. Other demulcents also can be included, such as those approved by the U.S. Food and Drug Administration (“US FDA”) and listed in 21 C.F.R. Part 349. They include hypromellose (0.2 to 2.5 percent), dextran 70 (0.1 percent when used with another polymeric demulcent listed in this regulation), gelatin (0.01 percent), liquid polyols, glycerin (0.2 to 1 percent), polyethylene glycol 300 or 400 (0.2 to 1 percent), propylene glycol (0.2 to 1 percent), polyvinyl alcohol (0.1 to 4 percent), povidone (or polyvinyl pyrrolidone, 0.1 to 2 percent). All compositions are in percent by weight of the total formulation, unless otherwise indicated.

In some other embodiments, a composition may include one or more emollients, such as those listed in 21 C.F.R. Section 349.14.

In a further aspect, the present invention provides a method for promoting wound healing, the method comprising applying to a wound a composition that comprises a bioadhesive polymer and a pharmaceutically acceptable vehicle, wherein the pharmaceutically acceptable vehicle comprises an aqueous medium.

In yet another aspect, the present invention provides a method for promoting wound healing, the method comprising applying to a wound any one composition herein disclosed.

In still another aspect, the present invention provides a method for promoting healing of an ocular wound, the method comprising applying to said ocular wound any one composition herein disclosed.

In still another aspect, the present invention provides a method for promoting healing of an ocular wound, the method comprising applying to said ocular wound any one composition herein disclosed, wherein said composition is in a form of an eye drop, suspension, emulsion, dispersion, or gel.

In still another aspect, the present invention provides a method for promoting healing of an ocular wound, the method comprising applying to said ocular wound any one composition herein disclosed, wherein said composition is in a form of an eye drop, emulsion, or gel.

In yet another aspect, the present invention provides a method for promoting healing of an ocular wound, the method comprising applying to said ocular wound any one composition herein disclosed; wherein said composition is in a form of an eye drop, suspension, emulsion, dispersion, or gel; and wherein said ocular wound results from an ocular surgery (such as cataract surgery or LASIK surgery).

In a further aspect, the present invention provides a method for promoting healing of an ocular wound, the method comprising applying to said ocular wound any one composition herein disclosed; wherein said composition is in a form of an eye drop, emulsion, or gel; and wherein said ocular wound results from cataract surgery.

### Experimental Testing

The current study was designed to investigate the effect of Besivance® on corneal reepithelialization in a rabbit wound healing model. For comparison purposes, the effects of Besivance were tested side-by-side with other approved ophthalmic fluoroquinolones, levofloxacin, gatifloxacin, and moxifloxacin. In addition, the effects of the Besivance vehicle with its preservative system, DuraSite®+benzalkonium chloride (BAK), and Azasite® (azithromycin ophthalmic solution 1%), another ophthalmic solution formulated in DuraSite®, on corneal reepithelialization were explored. In a separate study (BL08123/PH08198), two classical glucocorticoid ophthalmic solutions, Pred Mild® (prednisolone acetate ophthalmic suspension 0.12%) and Maxidex® (dexamethasone ophthalmic suspension 0.1%) significantly delayed corneal reepithelialization in this rabbit wound healing model, as expected, indicating that this is a valid animal model and can be used to evaluate the effects of pharmaceutical compounds on corneal wound healing.

Durasite® is an aqueous composition comprising polycarbophil (Noveon® AA-1), which is a poly(acrylic acid) lightly crosslinked with divinyl glycol.

To investigate the comparative effects of Besivance® (0.6% besifloxacin), Iquix® (1.5% levofloxacin), Zymar® (0.3% gatifloxacin), and Vigamox® (0.5% moxifloxacin) on corneal reepithelialization in the rabbit. In addition, the effects of Besivance® vehicle (Durasite®+100 ppm BAK), DuraSite®, BAK (benzalkonium chloride), and AzaSite® on corneal reepithelialization in this model were studied.

### Experimental Design

| Group | Drug/Brand |
|---|---|
| 1 | Saline (0.9% NaCl solution) |
| 2 | DuraSite ® |
| 3 | Besivance ® vehicle (DuraSite ® + 100 ppm BAK) |
| 4 | Besivance ® (besifloxacin hydrochloride ophthalmic |
| | suspension, 0.6%) |
| 5 | Iquix ® (levofloxacin ophthalmic solution 1.5%) |
| 6 | Zymar ® (gatifloxacin ophthalmic solution 0.3%; 50 ppm BAK) |
| 7 | Vigamox ® (moxifloxacin hydrochloride ophthalmic |
| | solution 0.5%) |
| 8 | 30 ppm BAK in saline |
| 9 | 100 ppm BAK in saline |
| 10 | 300 ppm BAK in saline |
| 11 | Azasite ® (azithromycin ophthalmic solution 1% in DuraSite ®; |
| | 30 ppm BAK) |

### Materials and Methods

Reagents. Besivance® (besifloxacin hydrochloride ophthalmic suspension 0.6%), DuraSite®, DuraSite® plus 100 ppm benzalkonium chloride (BAK), and 30, 100 or 300 ppm BAK in saline were prepared at Bausch & Lomb Incorporated (Rochester, N.Y.). Saline (preservative-free 0.9% NaCl; Demo, Athens, Greece or Hospira, Lake Forest, Ill.), Iquix® (levofloxacin ophthalmic solution 1.5%; Vistakon Pharmaceuticals, Jacksonville, Fla.), Zymar® (gatifloxacin hydrochloride ophthalmic solution 0.3%; Allergan, Irvine, Calif.), Vigamox® (moxifloxacin hydrochloride ophthalmic solution 0.5%; Alcon Laboratories, Fort Worth, Tex.), and AzaSite® (azithromycin ophthalmic solution 1%; Inspire, Durham, N.C.) were purchased from commercial sources and were used within the stated expiry date. All other reagents were purchased from standard commercial sources and were of the highest available quality and purity.

Animals. One hundred and ten pathogen-free New Zealand White rabbits were obtained from a commercial laboratory. Animals were at least 18 weeks old and 2-3 kg body weight at the time of treatment. Treatment of animals conformed to the American National Institutes of Health Principals of Laboratory Animal Care, the ARVO Statement for the Use of Animals in Ophthalmic and Vision Research, and specific institutional guidelines.

Pre-Treatment Examinations. Prior to placement on study, each animal underwent a pre-treatment ophthalmic examination (slit lamp with fluorescein staining). No signs of ocular irritation or other abnormalities were observed in any animal placed on study. Animals were then randomly assigned to eleven treatment groups (10 animals per group) according to a modified Latin square design. Animals were fasted at least 12 hours prior to corneal wound surgery.

Wounding Procedure. Prior to surgery, animals were anesthetized with an intravenous injection of 0.1 mL/kg of a ketamine/xylazine cocktail (77 mg/mL ketamine, 23 mg/mL xylazine). Proparacaine hydrochloride 0.5% (1-2 drops) was delivered to each right eye immediately prior to surgery. A standard epithelial wound was made in the center of each right eye by defining the area with a 9.5-mm trephine and then scraping the epithelium with a mini-blade to remove the full thickness epithelial layer¹⁶. Corneal wounds were not created in left eyes.

Topical Dosing. Following corneal wounding, the assigned test articles were instilled into both eyes of each animal 14 times over a 72-hour period. Animals were dosed at the following intervals: 15, 30, and 45 minutes, and 1, 2, 3, 6, 12, 18, 24, 36, 48, 60, and 72 hours post surgery, via ophthalmic dropper bottle at a volume of one drop per eye (35-50 μL).

Corneal Wound Evaluation. The corneal wound condition was evaluated immediately post wounding and at 12, 24, 36, 48, 60, and 72 hours. For each animal's wound evaluation, the right eye of the animal was stained with fluorescein and photographed (Topcon Imagenet EZ-Lite Photo Capture System; Paramus, N.J.) at a consistent focal distance. From the photographs, the perimeter of the wounds was determined using a Mechanical Polar Planimeter (LASISCO; Los Angeles, Calif.).

Histopathology. Following the final ocular evaluation (72 hours post-wounding), animals were euthanized with an intravenous overdose of Euthasol euthanasia solution. Both globes were collected from each animal and placed in 10% neutral buffered formalin. The samples were trimmed, processed, embedded, sectioned, and stained with hematoxylin and eosin. The central portion of each cornea was histologically evaluated for the signs of inflammation and corneal morphological changes. The tissues were evaluated without knowledge of the specific treatments received or formulation of the test articles.

### Data Analysis and Statistics

Numerical results were expressed as mean±SEM, n=10, unless indicated otherwise. Data were analyzed by a two-way ANOVA with repeated measures followed by the Tukey-Kramer test. In addition, overall effects were evaluated by analyzing integrated responses calculated as the areas under the curve (AUC) for each treatment over the time course. Data integration was conducted using the trapezoidal rule. Integrated responses were analyzed by a one-way ANOVA followed by the Tukey-Kramer test. Prior to statistical analysis, data were evaluated for normality and variance homogeneity and, if needed, results were subjected to Box-Cox transformations^{10, 11}. The specific transformations used are described in the legends to the figures. A p-value≦0.05 was predetermined as the criterion of statistical significance.

### Results:

Clinical Observation and Histopathology

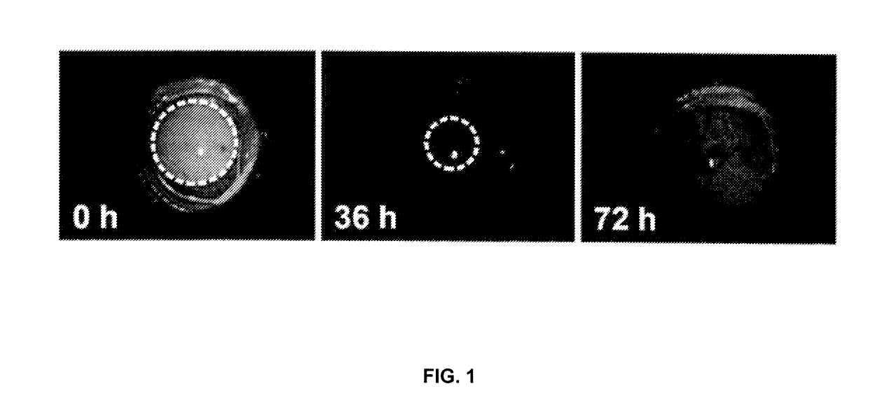

By 72 hours, most wounds were completely reepithelialized (≧95% compared to their 0-hour control) based on the lack of positive fluorescein staining (data not shown). FIG. 1 shows representative fluorescein-stained images of the corneal defect taken immediately after surgery, 36 hours post-surgery, and 72 hours post-surgery (left, middle and right panels, respectively) in an animal from the Besivance group. Hyperemia, chemosis, and discharge were noted in right eyes of all treatment groups during the first 36 hours post-surgery (data not shown). There were no significant findings in the unwounded left eye in all 110 rabbits (data not shown).

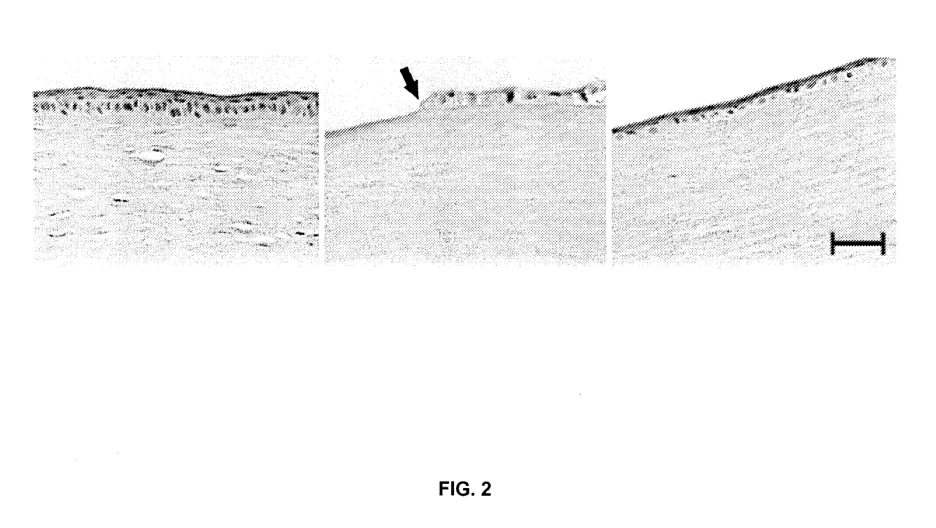

A cross section of the corneas was evaluated after hematoxylin and eosin staining. There were no significant histopathological findings in the unwounded left eye in all 110 rabbits in the study. The left panel of FIG. 2 shows an unwounded area of the cornea from a treated eye of an animal in the saline group, and it is histologically similar to that observed in the unwounded eyes. In wounded eyes with incomplete closure of the wound at 72 hours, the leading edge of the migrating corneal epithelium consisted of a single layer of cells (arrow in the middle panel of FIG. 2; from an animal in the Vigamox® group). In eyes with clinically complete closure of the wound, the reepithelialized layer of the wounded corneas generally demonstrated a thin epithelium as expected as part of the healing process and consisted of small basal cells with their long axes aligned parallel rather than perpendicular to the basement membrane. Covering these cells, there was a single layer of flat squamous cells without wing cells (FIG. 2, right panel; from an animal of Besivance® group)¹².

### Besivance® and Other Fluoroquinolones do not Affect Corneal Reepithelialization

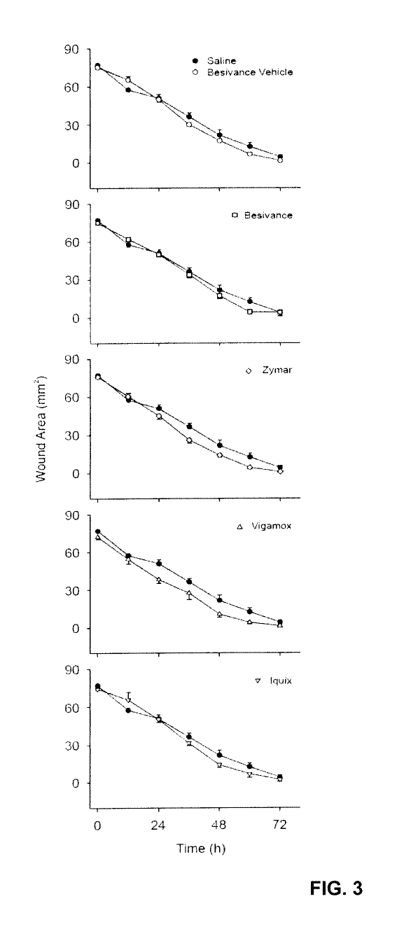

No significant effects on corneal reepithelialization time-courses were observed with Besivance®, Zymar®, Vigamox®, or Iquix® compared to the saline control at any of the time points examined (FIG. 3). The Besivance® vehicle (DuraSite®+100 ppm BAK; top panel) showed a similar reepithelialization time-course as the saline group, with no significant effect on corneal epithelialization at any of the time points examined.

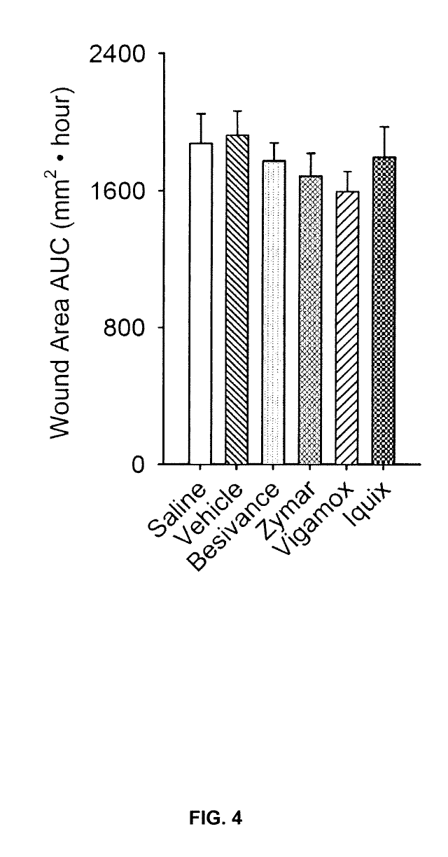

In addition, integrated responses were analyzed by calculating the areas under the curve (AUC) for each treatment over the time course from 0 to 72 hours. Again, no significant effects were seen when comparing the Besivance® vehicle, Besivance®, Iquix®, Zymar®, or Vigamox® to the saline control (FIG. 4). These data clearly indicate that fluoroquinolones tested did not affect corneal wound healing in this rabbit model.

### DuraSite® Accelerates Corneal Reepithelialization

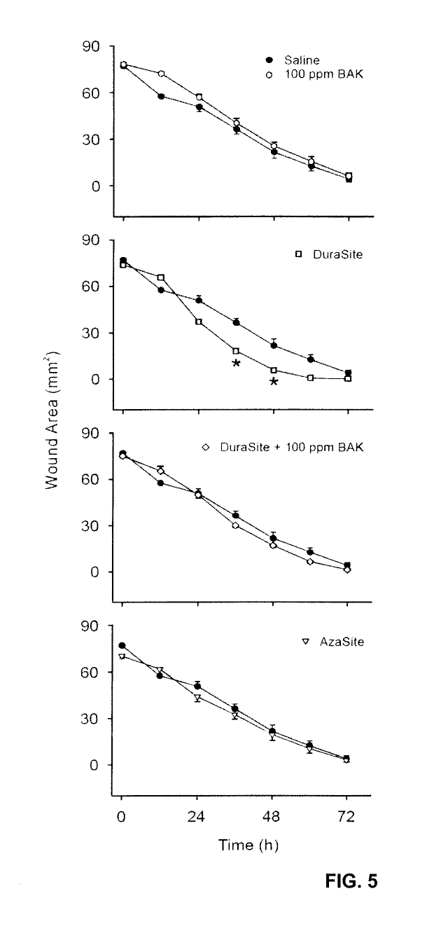

The effects of a number of the vehicle components and another anti-infective agent, AzaSite® also formulated in DuraSite® with 30 ppm BAK, were evaluated. Over the 72-hour observation period, DuraSite® alone (without BAK) showed the greatest reduction in mean wound area amongst all the treatment groups. This difference was statistically significant compared to the saline group at 36 and 48 hours post-surgery (middle panel in FIG. 5). AzaSite®, 100 ppm BAK in saline, and DuraSite®+100 ppm BAK had no statistically significant effect on the reepithelialized area at any of the time points examined compared to the saline control (FIG. 5). The effects of DuraSite® were also evident in the number of fully reepithelialized eyes at 72 hours post-surgery. In the DuraSite® group 9 out of 10 animals showed complete reepithelialization (90% incidence).

In the rest of the groups, 3-6 out of 10 animals did not fully reepithelialized at 72 hours post-surgery.

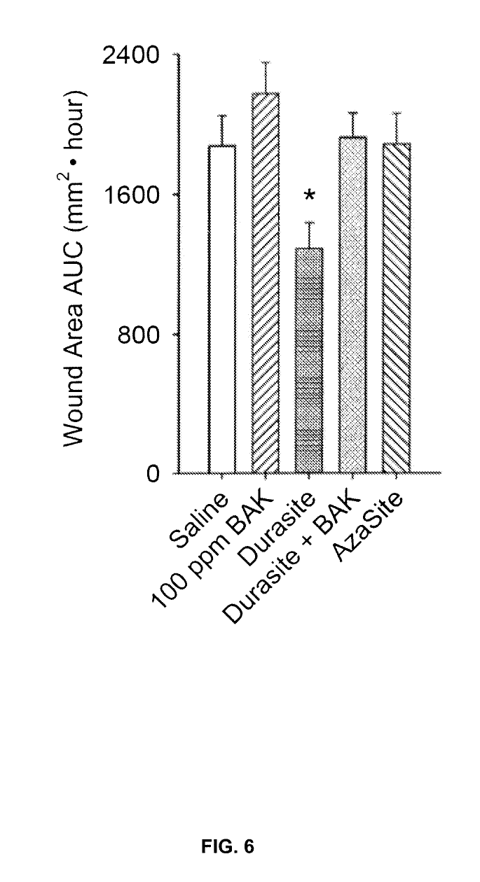

Integrated responses were analyzed by calculating the areas under the curve (AUC) for each treatment over the time course (0 to 72 hours). DuraSite® decreased the integrated wound area in a statistically significant manner when compared to the saline group, suggesting an increase in corneal reepithelialization compared to the saline control. No statistically significant effects were observed when comparing AzaSite, 100 ppm BAK in saline or DuraSite®+100 ppm BAK to the saline control (FIG. 6).

BAK does not Affect Corneal Reepithelialization

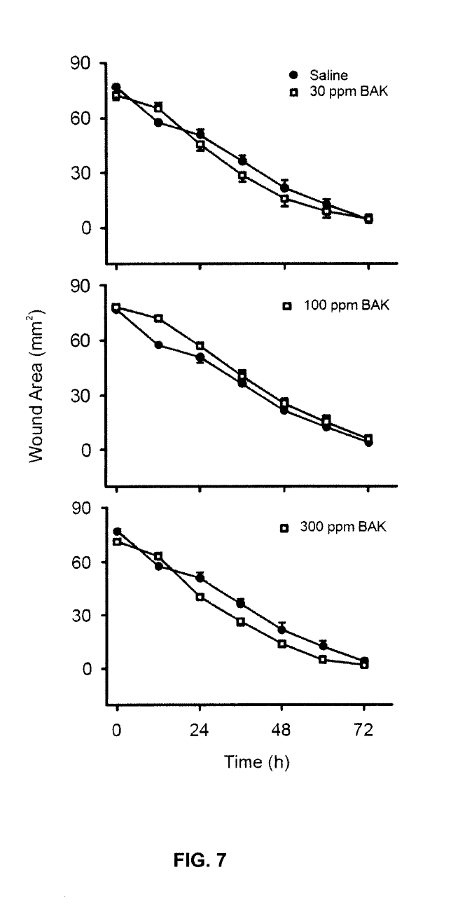

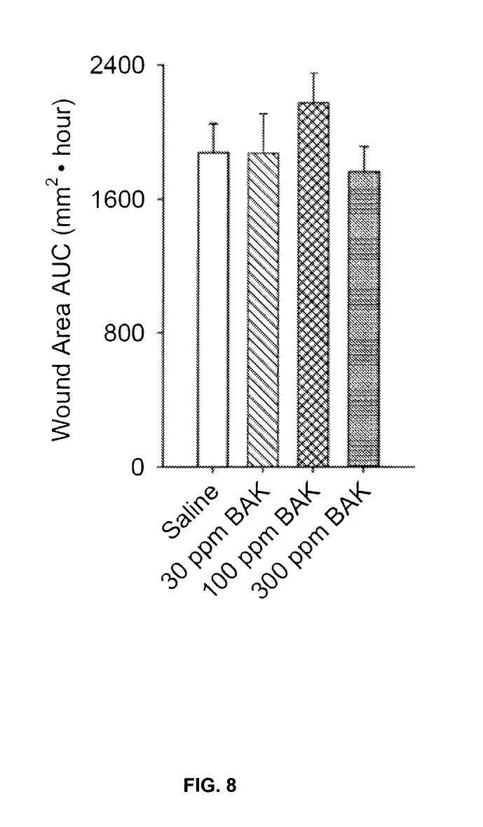

No significant effects on corneal reepithelialization time-courses were observed with any dose of BAK tested (30, 100, or 300 ppm in saline) compared to the saline control (FIG. 7). In addition, integrated responses were analyzed by calculating the areas under the curve (AUC) for each treatment over the time course from 0 to 72 hours. Again, no significant effects were seen when comparing all the BAK doses tested to the saline control (FIG. 8). These data suggest that BAK itself did not affect corneal wound healing in this rabbit model.

### Conclusions and Comments

Besivance®, besifloxacin hydrochloride ophthalmic suspension 0.6%, as well as the other three marketed fluoroquinolone ophthalmic solutions, Iquix®, Zymar® and Vigamox®, had no significant effect on corneal reepithelialization in the rabbit compared to the saline control.

Besivance® vehicle (DuraSite®, a mucoadhesive polymer-based system, +100 ppm BAK) and AzaSite® (azithromycin ophthalmic solution 1% formulated in DuraSite®+30 ppm BAK) had no significant effect on corneal reepithelialization in the rabbit compared to the saline control. Furthermore, DuraSite® alone (without 100 ppm BAK) accelerated corneal reepithelialization compared to the saline control. This effect was lost when Durasite® was combined with 100 ppm BAK, and the underlying mechanism remains unclear. The clinical significance of this observation on wound healing on the ocular surface and perhaps other tissues as well warrants further investigation.

BAK, a preservative included in Besivance vehicle at 100 ppm, had no significant effect at 30, 100 or 300 ppm on corneal reepithelialization in the rabbit compared to the saline control.

### REFERENCES

1. Broughton G., 2nd, Janis J. E., Attinger C. E., “The basic science of wound healing,” Plast. Reconstr. Surg. 2006; 117:12S-34S.
2. Zieske J. D., “Extracellular matrix and wound healing,” Curr. Opin. Ophthalmol. 2001; 12:237-241.
3. Imanishi J., Kamiyama K., Iguchi I., Kita M., Sotozono C., Kinoshita S., “Growth factors: importance in wound healing and maintenance of transparency of the cornea,” Prog. Retin. Eye Res. 2000; 19:113-129.
4. Saika S., Ohnishi Y., Ooshima A., Liu C. Y., Kao W. W., “Epithelial repair: roles of extracellular matrix.,” Cornea 2002; 21:S23-29.
5. Wilson S. E., Mohan R. R., Mohan R. R., Ambrosio R., Jr., Hong J., Lee J., “The corneal wound healing response: cytokine-mediated interaction of the epithelium, stroma, and inflammatory cells,” Prog. Retin. Eye Res. 2001; 20:625-637.
6. Lu L., Reinach P. S., Kao W. W., “Corneal epithelial wound healing,” Exp. Biol. Med. (Maywood) 2001; 226:653-664.
7. Kim M. J., Jun R. M., Kim W. K., et al., “Optimal concentration of human epidermal growth factor (hEGF) for epithelial healing in experimental corneal alkali wounds,” Curr. Eye Res. 2001; 22:272-279.
8. Brazzell R. K., Stern M. E., Aquavella J. V., Beuerman R. W., Baird L., “Human recombinant epidermal growth factor in experimental corneal wound healing,” Invest. Ophthalmol. Vis. Sci. 1991; 32:336-340.
9. Burling K., Seguin M. A., Marsh P., et al., “Effect of topical administration of epidermal growth factor on healing of corneal epithelial defects in horses,” Am. J. Vet. Res. 2000; 61:1150-1155.
10. Box G, Cox D. “An analysis of transformations,” J. Roy. Statist. Soc. Series B 1964; 26:211-252.
11. Box G., Hill W., “Correcting inhomogeneity of variance with power transformation weighting,” Technometrics 1974; 16:385-389.
12. Crosson C. E., Klyce S. D., Beuerman R. W., “Epithelial wound closure in the rabbit cornea. A biphasic process,” Invest. Ophthalmol. Vis. Sci. 1986; 27:464-473.

Non-limiting examples of embodiments of the present invention follow.

### EXAMPLE 1

The following ingredients are combined and mixed together until a substantially uniform mixture is obtained to produce a formulation suitable for promoting wound healing.

| Ingredient | % w/w |
|---|---|
| Glycerin | 0.6 |
| Sodium Alginate (Protanal LF200M) | 1 |
| Urea Hydrogen Peroxide | 0.1 |
| HAP (30%) | 0.05 |
| BHA (antioxidant) | 0.01 |
| Purified water | q.s. to 100 |

### EXAMPLE 2

The following ingredients are combined and mixed together until a substantially uniform mixture is obtained to produce a formulation suitable for accelerating wound healing.

| Ingredient | % w/w |
|---|---|
| Sodium Borate | 0.05 |
| Boric Acid | 0.6 |
| Glycerin | 0.75 |
| Propylene Glycol | 0.3 |
| Sodium Alginate (Protanal LF200M) | 0.3 |
| Urea Hydrogen Peroxide | 0.07 |
| HAP (30%) | 0.1 |
| BHA | 0.01 |
| Purified water | q.s. |

### EXAMPLE 3

The following ingredients are combined and mixed together until a substantially uniform mixture is obtained to produce a formulation suitable for promoting ocular wound healing.

| Ingredient | % w/w |
|---|---|
| Sodium Borate | 0.02 |
| Boric Acid | 0.5 |
| Glycerin | 0.6 |
| Propylene Glycol | 0.6 |
| Polycarbophil (Noveon ® AA-1, a cross- | 0.8 |
| linked poly(acrylic acid)) | |
| PHMB | 0.01 |
| EDTA disodium | 0.05 |
| Purified water | q.s. |

### EXAMPLE 4

The following ingredients are combined and mixed together until a substantially uniform mixture is obtained to produce a formulation suitable for promoting ocular wound healing.

| Ingredient | % w/w |
|---|---|
| Glycerin | 1 |
| Propylene Glycol | 0.2 |
| Carboxymethyl cellulose | 0.25 |
| Polyquaternium-1 (preservative) | 0.002 |
| Hydroxyalkyl phosphonate (HAP) | 0.05 |
| (30%) | |
| vitamin E TGPS | 0.05 |
| Phosphate buffer | q.s. to pH 6.3-6.8 |
| D-glucose | 0.5 |
| Purified water | q.s. |

### EXAMPLE 5

The following ingredients are combined and mixed together until a substantially uniform mixture is obtained to produce a formulation suitable for promoting ocular wound healing.

| Ingredient | % w/w |
|---|---|
| Sodium Borate | 0.03 |
| Boric Acid | 0.35 |
| Propylene Glycol | 0.6 |
| HPMC | 0.4 |
| EDTA disodium | 0.05 |
| Benzalkonium chloride | 0.01 |
| Carbopol ® 974P NF | 0.5 |
| Sucrose | 0.5 |
| Phosphate buffer | q.s. to pH 6.0-7.0 |
| Purified water | q.s. |

### EXAMPLE 6

The following ingredients are combined and mixed together until a substantially uniform mixture is obtained to produce a formulation suitable for promoting wound healing.

| Ingredient | % w/w |
|---|---|
| Sodium Borate | 0.06 |
| Boric Acid | 0.7 |
| Propylene Glycol | 0.6 |
| Hyaluronic acid | 0.25 |
| Polyquaternium-42 | 0.005 |
| Gallic acid | 0.05 |
| Phosphate buffer | q.s. to 7.0-7.6 |
| Purified water | q.s. |

### EXAMPLE 7

The following ingredients are combined and mixed together until a substantially uniform mixture is obtained to produce a formulation suitable for promoting ocular wound healing.

| Ingredient | % w/w |
|---|---|
| Sodium Borate | 0.06 |
| Boric Acid | 0.7 |
| Glycerin | 0.6 |
| Propylene Glycol | 0.6 |
| HPMC | 0.4 |
| Sodium perborate 4H₂O | 0.07 |
| Cinnamic acid | 0.05 |
| EDTA disodium | 0.05 |
| HAP (30%) | 0.02 |
| Polycarbophil Noveon ® AA-1 | 0.5 |
| Maltose | 1 |
| Phosphate buffer | q.s. to pH 7-7.5 |
| Purified water | q.s. |

### EXAMPLE 8

The following ingredients are combined and mixed together until a substantially uniform mixture is obtained to produce a formulation suitable for promoting wound healing.

| Ingredient | % w/w |
|---|---|
| Sodium Borate | 0.06 |
| Boric Acid | 0.7 |
| Glycerin | 0.6 |
| Propylene Glycol | 0.6 |
| Polycarbophil Noveon ® AA-1 | 0.5 |
| Sodium perborate monohydrate | 0.1 |
| Sodium perborate tetrahydrate | 0.05 |
| Vitamin E TPGS | 0.1 |
| HAP (30%) | 0.1 |
| Carbopol ® 934P NF | 0.3 |
| D-glucose | 0.5 |
| Phosphate buffer | q.s. to pH 6.3-7.4 |
| Purified water | q.s. |

### EXAMPLE 9

The following ingredients are combined and mixed together until a substantially uniform mixture is obtained to produce a formulation suitable for promoting ocular wound healing.

| Ingredient | % w/w |
|---|---|
| Sodium Borate | 0.06 |
| Boric Acid | 0.7 |
| Glycerin | 0.8 |
| Propylene Glycol | 0.4 |
| Carboxymethyl cellulose | 0.25 |
| Polyquaternium-1 (PQ-1) | 0.001 |
| Ascorbic acid | 0.1 |
| HAP (30%) | 0.1 |
| BHA | 0.02 |
| Besifloxacin monohydrochloride | 0.3 |
| Phosphate buffer | q.s. to pH 6.5-7.5 |
| D-glucose | 0.6 |
| Purified water | q.s. |

### EXAMPLE 10

The following ingredients are combined and mixed together until a substantially uniform mixture is obtained to produce a formulation suitable for promoting ocular wound healing.

| Ingredient | % w/w |
|---|---|
| Sodium Borate | 0.06 |
| Boric Acid | 0.7 |
| Glycerin | 0.6 |
| Polycarbophil Noveon ® AA-1 | 0.6 |
| Sodium hyaluronate | 0.25 |
| PQ-1 | 0.1 |
| Resveratrol | 0.075 |
| Ascorbic acid | 0.025 |
| HAP (30%) | 0.1 |
| Loteprednol Etabonate | 0.5 |
| D-glucose | 0.5 |
| Potassium chloride | 0.2 |
| Phosphate buffer | q.s. to pH 6.5-7.5 |
| Purified water | q.s. |

While specific embodiments of the present invention have been described in the foregoing, it will be appreciated by those skilled in the art that many equivalents, modifications, substitutions, and variations may be made thereto without departing from the spirit and scope of the invention as defined in the appended claims.

What is claimed is:

1. A composition comprising a bioadhesive polymer and a pharmaceutically acceptable vehicle, wherein the bioadhesive polymer comprises a mucoadhesive adhesive, and the pharmaceutically acceptable vehicle comprises a liquid medium, wherein the composition promotes healing of a wound.

2. The composition of any one of previous claims, wherein the liquid medium comprises an aqueous medium.

3. The composition of any one of previous claims, wherein the bioadhesive polymer is suspended or dissolved in the aqueous medium, and wherein the bioadhesive polymer is selected from the group consisting of hydrophilic polymers, hydrogels, and mixtures thereof.

4. The composition of any one of previous claims, wherein the bioadhesive polymer is selected from the group consisting of polyvinylpyrrolidone, methyl cellulose, carboxymethyl cellulose and its salts, hydroxypropylmethyl cellulose, other hydrophilic cellulose derivatives, alginate and its salts, hyaluronic acid and its salts, and chitosan and derivatives thereof.

5. The composition of any one of previous claims, wherein the amount of the bioadhesive polymer is in the range from about 0.001 to about 10 percent by weight of the total composition.

6. The composition of any one of previous claims, wherein the bioadhesive polymer is suspended or dissolved in the aqueous medium; and the bioadhesive polymer comprises a lightly crosslinked poly(acrylic acid) in an amount in the range from about 0.1 to about 7 percent by weight of the total composition.

7. The composition of any one of previous claims, wherein the crosslinked poly(acrylic acid) is selected from the group consisting of polycarbophil (poly(acrylic acid) crosslinked with divinyl glycol), Carpobol® polymers (poly(acrylic acid) crosslinked with allyl ethers of pentaerythritol or allyl ethers of sucrose), and poly(C₁₀₋₃₀ alkyl acrylate/acrylic acid) crosspolymers.

8. The composition of any one of previous claims, wherein the composition as disclosed herein has a viscosity in the range from about 2 to about 2,000 centipoises (or mPa·s), as measured by a Brookfield viscometer (Model RVDV Ill) at 25° C. and a shear rate of 1-7 sec⁻¹, with a CPE-40 spindle.

9. The composition of any one of previous claims; wherein the bioadhesive polymer comprises a polycarbophil in an amount in the range from about 0.1 to about 7 percent by weight of the total composition; the composition has a viscosity in the range from about 3 to about 1500 mPa·s.

10. The composition of any one of previous claims, wherein the composition comprises: (a) a bioadhesive polymer; (b) a non-ionic or ionic tonicity agent; (c) a pharmaceutically acceptable preservative; and (d) a pharmaceutically acceptable aqueous medium; wherein the bioadhesive polymer is suspended or dissolved in the aqueous medium; and the bioadhesive polymer comprises a polycarbophil in an amount in the range from about 0.1 to about 7 percent by weight of the total composition; wherein the composition has a viscosity in the range from about 3 to about 1500 mPa·s.

11. The composition of any one of previous claims, wherein the composition comprises: (a) a bioadhesive polymer; (b) a tonicity agent (non ionic or inonic); (c) a pharmaceutically acceptable preservative; (d) a preservative-enhancing material; and (e) a pharmaceutically acceptable aqueous medium; wherein the bioadhesive polymer is suspended or dissolved in the aqueous medium; and the bioadhesive polymer comprises a polycarbophil in an amount in the range from about 0.1 to about 7 percent by weight of the total composition; wherein the composition has a viscosity in the range from about 3 to about 1500 mPa·s; wherein the preservative-enhancing material is selected from the group consisting of D-glucose, sucrose, maltose, D-mannose, trehalose, glutamic acid, mixtures thereof, and combinations thereof.

12. The composition of any one of previous claims, wherein the composition comprises: (a) a bioadhesive polymer; (b) a non ionic or inonic tonicity agent; (c) a pharmaceutically acceptable preservative; (d) a preservative-enhancing material; (e) a chelating agent; and (f) a pharmaceutically acceptable aqueous medium; wherein the bioadhesive polymer is suspended or dissolved in the aqueous medium; and the bioadhesive polymer comprises a polycarbophil in an amount in the range from about 0.1 to about 7 percent by weight of the total composition; wherein the composition has a viscosity in the range from about 3 to about 1500 mPa·s, as measured at the condition disclosed herein; wherein the preservative-enhancing material is selected from the group consisting of D-glucose, sucrose, maltose, D-mannose, trehalose, glutamic acid, mixtures thereof, and combinations thereof.

13. The composition of any one of previous claims, wherein the composition comprises: (a) a bioadhesive polymer; (b) a non ionic or inonic tonicity agent; (c) a pharmaceutically acceptable preservative; (d) a preservative-enhancing material; (e) a chelating agent; and (f) a pharmaceutically acceptable aqueous medium; wherein the bioadhesive polymer is suspended or dissolved in the aqueous medium; and the bioadhesive polymer comprises chitosan or a derivative thereof, in an amount in the range from about 0.1 to about 7 percent by weight of the total composition; wherein the composition has a viscosity in the range from about 3 to about 1500 mPa·s, as measured at the condition disclosed herein; wherein the preservative-enhancing material is selected from the group consisting of D-glucose, sucrose, maltose, D-mannose, trehalose, glutamic acid, mixtures thereof, and combinations thereof.

14. The composition of any one of the previous claims, further comprising an active pharmaceutical ingredient (or therapeutic agent) selected from the group consisting of anti-inflammatory agents, antibiotics, immunosuppressive agents, antiviral agents, antifungal agents, antiprotozoal agents, combinations thereof, or mixtures thereof.

15. A method of for promoting wound healing, the method comprising applying to a wound a composition of any one of the previous claims.

16. The method of claim 15, wherein said wound is an ocular wound, and wherein the method comprises applying to said ocular wound any one composition of claims 1-14.

17. The method of any one of claims 15-16, wherein said composition is in a form of an eye drop, suspension, emulsion, dispersion, or gel.

18. The method of any one of claims 15-17, wherein said composition is in a form of an eye drop, emulsion, or gel.

19. The method of any one of claims 15-18, wherein said wound is an ocular wound resulting from an ocular surgery.

20. The method of any one of claims 15-19, wherein said wound is an ocular wound resulting from cataract surgery.
